(19) United States
(12) Patent Application Publication
(10) Pub. No.: US 2007/0117794 A1
(43) Pub. Date: May 24, 2007

(54) Methods of treatment using oxytocin receptor agonists

(75) Inventors: Zia Rahman, Princeton, NJ (US); Lynn Resnick, Parlin, NJ (US); Sharon Joy Rosenzweig-Lipson, East Brunswick, NJ (US); Robert Hamlink Ring, Yardley, PA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/584,995

(22) Filed: Oct. 23, 2006

Related U.S. Application Data
(60) Provisional application No. 60/729,656, filed on Oct. 24, 2005.

(51) Int. Cl. A61K 31/551

(52) U.S. Cl. 514/220

(57) ABSTRACT

Methods for treating and preventing anxiety, anxiety-related disorders, schizophrenia and schizophrenia-related disorders are described herein wherein said methods comprise the administration of oxytocin receptor agonists.

### CROSS REFERENCE TO RELATED APPLICATIONS

This application claims priority from copending Provisional Application No. 60/729,656, filed on Oct. 24, 2005, the entire disclosure of which is hereby incorporated by reference.

### FIELD OF THE INVENTION

The present invention relates to the use of non-peptide oxytocin receptor agonists for the treatment of schizophrenia, schizophrenia-related disorders, anxiety and anxiety-related disorders.

### BACKGROUND OF THE INVENTION

Oxytocin (OT) is a nonapeptide, differing in two amino acids from its sister neurohypophyseal peptide, arginine vasopressin (AVP). OT is synthesized principally in two divisions of hypothalamic neurons, the magnocellular cells of the supraoptic (SON) and paraventricular (PVN) nuclei, and the parvocellular cells of the PVN. The oxytocinergic neurons of the SON project to the posterior pituitary where OT is released into the peripheral circulation from axon terminals in the bed capillaries. This peripheral release is most familiarly associated with the OT effects in women during the peripartum period; when OT participates in stimulating uterine smooth muscle contraction during labor, and the triggering of the milk ejection reflex in mammary myoepithelial cells during lactation. Although Sir Henry Dale first described the uterotonic effects of oxytocin back in 1909, it was not until the 1980s that modern obstetrics began exploiting its tocogenic activity to help optimize delivery without disastrous side effects. Intriguingly however, studies with TO knock-out mice have demonstrated that OT is not essential for normal parturition which helps to illustrate the teleological importance of its other, perhaps lesser appreciated, role in regulating central nervous system (CNS) function(s). (Young, W. S., 3rd et al. Targeted reduction of oxytocin expression provides insights into its physiological roles. Adv Exp Med Biol 449, 231-40 (1998)).

Only recently (since the early 1990's) has the distinction between the central and peripheral oxytocinergic systems has been appreciated. Following cloning of the oxytocin receptor (OTR) and numerous immunolocalization and radioligand binding studies, many were surprised to find the extent to which oxytocinergic efferents, particularly emanating from the PVN, innvervate extrahypothalamic sites throughout the CNS. Collectively, this network of connections form what is referred to as the central oxytocinergic system, which positions OT to exert influence on key neuroanatomical substrates behind social recognition (olfactory bulb), aggression/avoidance (MPOA), motivation (NA/DA, brainstem nuclei), and fear/anxiety behavior (amygdala, hypothalamus, BNST). Although emerging evidence has extended roles for oxytocin to include involvement in memory and nociception, the majority of CNS research has focused on OT involvement in socio-sexual/reproductive behaviors (e.g. sexual behavior, parental behavior, pair-bond formation). A unifying principle of oxytocinergic action in the CNS begins to emerge: OT facilitates social interaction by reducing the anxiety associated with such encounters. (McCarthy, M. M. Estrogen modulation of oxytocin and its relation to behavior. Adv Exp Med Biol 395, 235-45 (1995)).

A commonly observed consequence of friendly social contact is the induction of a psychophysiological pattern involving sedation, relaxation, decreased sympathoadrenal activity, and increased vagal tone which is in contrast to fear/anxiety which leads to general mental activation, locomotor activity, and catabolic activity. (Uvnas-Moberg, K. Oxytocin linked antistress effects—the relaxation and growth response. Acta Physiol Scand Suppl 640, 38-42 (1997)). Evidence consistently implicates the central oxytocinergic system as a key axis on which these opposing effects are mediated.

Effects of the peptide oxytocin itself in models of CNS activity have been noted. For example:

1. OT (1-4 ug/kg), administered sub-cutaneously (s.c.), produces a decrease in peripheral locomotor activity in the Open-Field model of anxiety, indicative of an anxiolytic-like effect. (Uvnas-Moberg, K., Ahlenius, S., Hillegaart, V. & Alster, P. High doses of oxytocin cause sedation and low doses cause an anxiolytic-like effect in male rats. Pharmacol Biochem Behav 49, 101-6 (1994)).

2. OT (3 mg/kg), administered intraperetonially (i.p.), produced anxiolytic-like activity elevated-plus maze. (McCarthy, M. M., McDonald, C. H., Brooks, P. J. & Goldman, D. An anxiolytic action of oxytocin is enhanced by estrogen in the mouse. Physiol Behav 60, 1209-15 (1996)).

3. OT (10-100 ng), administered intracerebroventricularly (i.c.v.), produced increases in open arm entries and time spent in the open arms of the elevated plus-maze, suggesting that OT exerts a centrally-mediated anxiolytic-like effect. (Windle, R. J., Shanks, N., Lightman, S. L. & Ingram, C. D. Central oxytocin administration reduces stress-induced corticosterone release and anxiety behavior in rats. Endocrinology 138, 2829-34 (1997)).

4. Anxiolytic activity of OT in the elevated-plus maze of pregnant or lactating rats, but not virgin rats, suggesting a role for estrogen in the control of OT. (Neumann, I. D., Torner, L. & Wigger, A. Brain oxytocin: differential inhibition of neuroendocrine stress responses and anxiety-related behaviour in virgin, pregnant and lactating rats. Neuroscience 95, 567-75 (2000)).

5. Enhanced anxiety behavior in the elevated plus maze is observed in female OT knock-out mice. (Mantella, R. C., Vollmer, R. R., Li, X. & Amico, J. A. Female oxytocin-deficient mice display enhanced anxiety-related behavior. Endocrinology 144, 2291-6 (2003). 6. OT is known to inhibit CRF release, and cause a down-regulation of the hypothalamic-pituitary-adrenal (HPA) axis. (Neumann, I. D., Wigger, A., Torner, L., Holsboer, F. & Landgraf, R. Brain oxytocin inhibits basal and stress-induced activity of the hypothalamo-pituitary-adrenal axis in male and female rats: partial action within the paraventricular nucleus. J Neuroendocrinol 12, 235-43 (2000)). Hyperactivity of the HPA axis, often linked to increased corticotrophin-releasing factor (CRF)-mediated ACTH release, is commonly observed in human depressed patients.

7. In humans, deceased levels of anxiety and the incidence of anxiety-related disorders are observed in humans during lactation, a physiological process mediated by increased levels of OT (Altemus, Neuropeptides in anxiety disorders. Effects of lactation. Ann N Y Acad Sci 771: 697-707 (1995).

8. Both acute and chronic treatment of adult male rats with the SSRI citalopram (20 mg/kg) led to an increase in plasma levels of oxytocin, suggesting that oxytocin release may be an import aspect of the pharmacological actions of antidepressants. (Uvnas-Moberg, K., Bjokstrand, E., Hillegaart, V. & Ahlenius, S. Oxytocin as a possible mediator of SSRI-induced antidepressant effects. Psychopharmacology (Berl) 142, 95-101 (1999)).

The biological activity of OT is mediated by a family of four receptors that include, in addition to the specific oxytocin receptor, OTR, all known vasopressin receptors (V1a( V1R), V2( V2R), V1b( V3R)). The OTR is a class V G-protein coupled receptor (GPCR) that exhibits its highest sequence similarity with the V3R. Consistent with sequence similarities of this family; only a 10-fold higher selectivity exists for OT compared to AVP at the OTR. (Chini, B. et al. Two aromatic residues regulate the response of the human oxytocin receptor to the partial agonist arginine vasopressin. FEBS Lett 397, 201-6 (1996); Postina, R., Kojro, E. & Fahrenholz, F. Separate agonist and peptide antagonist binding sites of the oxytocin receptor defined by their transfer into the V2 vasopressin receptor. J Biol Chem 271, 31593-601 (1996)). Expression of the oxytocin receptor (OTR) is observed throughout the CNS with notable differences in distribution patterns between species. (Tribollet, E., Dubois-Dauphin, M., Dreifuss, J. J., Barberis, C. & Jard, S. Oxytocin receptors in the central nervous system. Distribution, development, and species differences. Ann N Y Acad Sci 652, 29-38 (1992)). A common feature OTR expression across species is the robust expression in the limbic system. In rodents, OT binding sites are found in the bed nucleus of the stria terminalis (BSNT), central amygdaloid nucleus, ventromedial hypothalamic nucleus, and ventral subiculum. The pattern of OT binding is quite different in humans, but consistent with a proposed role in the regulation of social behaviors, with strong binding observed in the lateral septal nucleus and basal nuclei of Meynert which provides direct cholinergic input to the basolateral amygdaloid nucleus. (Loup, F., Tribollet, E., Dubois-Dauphin, M. & Dreifuss, J. J. Localization of high-affinity binding sites for oxytocin and vasopressin in the human brain. An autoradiographic study. Brain Res 555, 220-32 (1991)).

In addition to the significant amount of evidence linking oxytocin signaling with anxiolytic effects in mammals, there is also at least some evidence linking oxytocin signaling with schizophrenia. For example, there have been numerous studies that have indicated that perturbations in oxytocin concentration have been found in schizophrenic patients and that treatment of schizophrenics with neuroleptics can further increase oxytocin concentrations. (Beckmann, H., Lang, R. E., Gattaz, W. F. Vasopressin-oxytocin in cerebrospinal fluid of schizophrenic patients and normal controls. Psychoneuroendocrinology 10: 187-191). In a rat model of prepulse inhibition (inhibition of the startle reflex by immediately preceding an intense stimulation with a lesser-intensity stimulation), it has been demonstrated that subcutaneous administration of oxytocin can dose-dependently restore prepulse inhibition induced by dizocilpine (a non-competitive NMDA-antagonist) and amphetamine. Decreased prepulse inhibition has been demonstrated for patients with schizophrenia and it has been hypothesized that oxytocin action on this parameter is indicative of an antipsychotic action, since such prepulse inhibition activity is strongly correlated with antipsychotic drug activity. (Feifel, D., and Reza, T. Oxytocin modulates psychotomimetic-induced deficits in sensorimotor gating. Psychopharmacology 141: 93-98 (1999)).

The discovery of new methods for the treatment and prevention of anxiety and schizophrenia are of paramount importance given the severe implications that each of these disorders can represent as well as the large number of people who are not presently being treated in a satisfactory manner. Given that oxytocin has been implicated in the treatment of anxiety and schizophrenia, there is a strong need for the discovery of new methods for treating anxiety and schizophrenia where these new methods would employ not oxytocin itself, but rather non-peptide agonists for the oxytocin receptor. Such compounds could present opportunities for varied modulation of the oxytocin receptor, thus increasing the possibilities for clinical success. Furthermore, such compounds could present the added advantage of improved pharmaceutical properties, for example, by rendering themselves available upon oral administration and/or having increased central-mediated effects. The present invention describes, herein for the first time, methods of treating and preventing anxiety and schizophrenia using certain non-peptide oxytocin receptor agonists.

### SUMMARY OF THE INVENTION

The present invention describes methods of treating schizophrenia and schizophrenia-related disorders, anxiety and anxiety-related disorders comprising the administration to a mammal a compound of formula 1 or a pharmaceutically acceptable salt thereof:

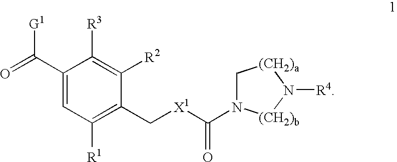

The present invention also describes methods of treating schizophrenia, schizophrenia-related disorders, anxiety, and anxiety-related disorders comprising the administration to a mammal of a compound of formula 2 or a pharmaceutically acceptable salt thereof:

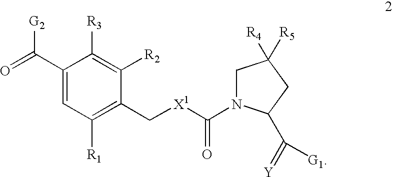

### DETAILED DESCRIPTION OF THE INVENTION

### BRIEF DESCRIPTION OF THE DRAWINGS

FIG. 1—“Anxiolytic-like effects of Oxytocin in the mouse four-plate model. Central administration of Oxytocin (1-10 μg, icv, 30 minutes prior to testing) produced a dose-dependent increase in punished crossings, suggesting an anxiolytic-like effect. *p<0.05 compared to vehicle (Veh) group, n=10 per group.”

FIG. 2—“Anxiolytic-like effects of the non-peptide oxytocin receptor agonist Cpd A in the mouse four-plate model. Peripheral administration of cpd A (3-100 mg/kg, ip, 30 minutes prior to testing) increases the number of punished crossings, suggesting an anxiolytic-like effect. *p<0.05 compared to vehicle (Veh) group, n=10 per group.”

FIG. 3—“The non-peptide oxytocin receptor antagonist Cpd B dose-dependently blocks the anxiolytic-like effect of Cpd A in the four-plate model. Cpd A (10 mg/kg, ip, 30 minutes prior to testing) increases punished crossings, which is blocked by co-administration of Cpd B (10-30 mg/kg, ip, 30 minutes prior to testing). *p<0.05 compared to Vehicle (Veh); ** p<0.05 compared to cpd A, n=10 per group.”

FIG. 4—“Effects of Cpd A on MK801 disrupted PPI and Startle Response in Rats. MK801 (0.1 mpk, sc. 10 mins pretreatment) produced significant disruption across aft three prepulse intensities tested. Cpd A (3, 10, 30 mg/kg i.p., 30 mins prior to test) reversed MK801—induced deficit at 10 dB and 15 dB. * MK801 produced significant disruption across all three prepulse intensities •30 mg/kg reversed MK801 induced disruption at 10 dB and 15 dB”

FIG. 5—“Effects of Cpd A (HCl salt) on d-Amphetamine induced disrupted PPI and Startle Response in Rats. d-Amphetamine (4 mg/kg, sc. 10 mins pretreatment) produced significant disruption across all three prepulse intensities tested. Cpd A (HCl salt) (10 mg/kg ip, 30 mins prior to test) reversed d-amphetamine—induced deficit at 5 dB and 10 dB. Cpd A (HCl salt) (30 mg/kg ip, 30 mins prior to test) reversed d-amphetamine induced disruption at 10 dB. * d-Amphetamine produced significant disruption across all three prepulse intensities #10 mg/kg reversed amphetamine induced disruption at 5 dB and 30 mg/kg reversed amphetamine induced disruption at 10 dB and 15 dB”

In some embodiments, this invention describes a method of treating schizophrenia or a schizophrenia-related disorder, anxiety and anxiety-related disorders comprising the administration to a mammal a compound of formula 1, or a pharmaceutically acceptable salt thereof: 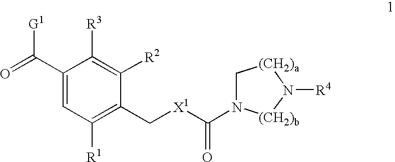
wherein:
G¹, R¹, R², R³, R⁴, X¹, a, and b are as defined in WO03/016316 (page 63-65; claim 1) which is herein incorporated by reference in its entirety.

In some embodiments, for the compound of formula 1, G¹ is 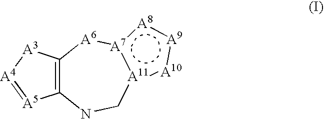
wherein A₃ is S; NH; N—C₁₋₃alkyl; —CH═CH— or CH═N; A₄ is CH; A₅ is CH; A₆ is NH; A₇ is C; A₈ is N—(CH₂)_{d}-R₇; A₉ is N; A₁₀ is CH and A₁₁ is C; wherein d is 1, 2 or 3; and R₇ is selected from hydrogen; C₁₋₃ alkyl; optionally substituted phenyl; OH; O-alkyl; O-acyl; S-alkyl; NH₂; NH-alkyl; N(alkyl)₂; NH-acyl; N(alkyl)-acyl; CO₂H; CO₂-alkyl; CONH₂; CONH-alkyl; CON(alkyl)₂; CN; and CF₃.

In some embodiments, for the compound of formula 1, G¹ is

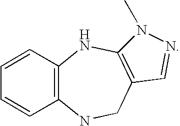

In some embodiments, for the compound of formula 1, R¹, R² and R³ are each independently selected from hydrogen; alkyl; Fl; or Cl.

In some embodiments, for the compound of formula 1, R₄ is selected from

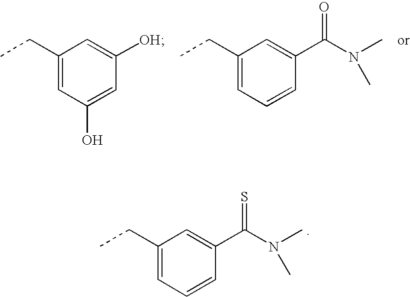

In certain aspects, for the compound of formula 1; two of R¹, R² and R³ are hydrogen and the other is not hydrogen.

In some embodiments, for the compound of formula 1, R¹ and R³ are each hydrogen, and R² is methyl.

In some embodiments, for the compound of formula 1; R₄ is

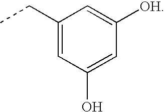

In certain aspects, the compound of formula 1 is: 4,-(3,5-Dihydroxy-benzyl)-piperazine-1-carboxylic acid 2-methyl-4-(3-methyl-4,10-dihydro-3H-2,3,4,9-tetra-aza-benzo[f]azulene-9-carbonyl)-benzylamide; 4,-(3,5-Dihydroxy-benzyl)-piperazine-1-carboxylic acid 2,6-dimethyl-4-(3-methyl-4,10-dihydro-3H-2,3,4,9-tetra-aza-benzo[f]azulene-9-carbonyl)-benzylamide; 4,-(3,5-Dihydroxy-benzyl )-piperazine-1-carboxylic acid 3-chloro-4-(3-methyl-4,10-dihydro-3H-2,3,4,9-tetra-aza-benzo[f]azulene-9-carbonyl)-benzylamide; 4,-(3,5-Dihydroxy-benzyl)-piperazine-1-carboxylic acid 2-fluoro-4-(3-methyl-4,10-dihydro-3H-2,3,4,9-tetra-aza-benzo[f]azulene-9-carbonyl)-benzylamide; 4,-(3-Dimethylcarbamoyl-benzyl)-piperazine-1-carboxylic acid 2-methyl-4-(3-methyl-4,10-dihydro-3H-2,3,4,9-tetra-aza-benzo[f]azulene-9-carbonyl)-benzylamide; and 4,-(3-Dimethylthiocarbamoyl-benzyl)-piperazine-1-carboxylic acid 2-methyl-4-(3-methyl-4,10-dihydro-3H-2,3,4,9-tetra-aza-benzo[f]azulene-9-carbonyl)-benzylamide; or a pharmaceutically acceptable salt thereof.

In some embodiments, the compound of formula 1 is administered with at least one pharmaceutically acceptable excipient.

In some embodiments, the compound of formula 1 is administered to a human.

In some embodiments, this invention is directed to the treatment of schizophrenia, the treatment of schizophrenia-related disorders, and the treatment of anxiety and anxiety-related disorders, using compounds of formula 2, and pharmaceutically acceptable salts thereof; 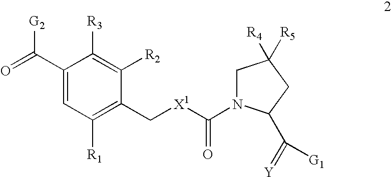
wherein G₁, R₁, R₂, R₃, X₁, R₄, R₅, Y, and G₂ are all as described in WO 03/000692 (page 61-65, claim 1), which reference is herein incorporated by reference in its entirety.

In certain embodiments, for the compound of formula 2:
G₂ is (II) 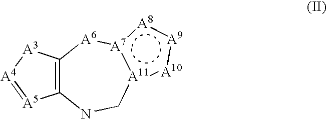
wherein A₃ is S; NH; N—C₁₋₃alkyl; —CH═CH— or CH═N; A₄ is CH; A₅ is CH; A₆ is NH; A₇ is C; A₈ is N—(CH₂)_{d}-R₇; A₉ is N; A₁₀ is CH and A₁₁ is C; wherein d is 1, 2 or 3; and R₇ is selected from hydrogen; C₁₋₃ alkyl; optionally substituted phenyl; OH; O-alkyl; O-acyl; S-alkyl; NH₂; NH-alkyl; N(alkyl)₂; NH-acyl; N(alkyl)-acyl; CO₂H; CO₂-alkyl; CONH₂; CONH-alkyl; CON(alkyl)₂; CN; and CF₃.
R₁, R₂, and R₃ are each independently selected from the group consisting of hydrogen; alkyl; O-alkyl; Fl; Cl; or Br;
X₁ is NH or O;
R₄ and R₅ are each independently selected from the group consisting of hydrogen; O-alkyl; O-benzyl; and F; or R₄ and R₅ together are =O; —O(CH₂)ₐO—; or
—S(CH₂)ₐS—;
a is 2 or 3;
Y is O or S; and
G₁ is 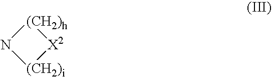
wherein h is 1 or 2; I is 1, 2 or 3; and X₂ is N-alkyl.

In some embodiments, for the compound of formula 2, G₂ is:

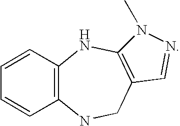

In some embodiments, for the compound of formula 2, two of R¹, R² and R³ are hydrogen and the other is not hydrogen.

In some embodiments, for the compound of formula 2, X₁ is NH.

In some embodiments, for the compound of formula 2, R₄ and R₅ are each independently selected from hydrogen and O-alkyl.

In some embodiments, for the compound of formula 2, G₁ is 1-Methyl-[1,4]diazepane.

In certain embodiments, the compound of formula 2 is: 4-methyl-1-(N-(2-methyl-4-(2,3,4,5-tetrahydro-1,5-benzodiazepin-4-on-1-yl-carbonyl)benzylcarbamoyl)-L-thioprolyl)perhydro-1,4-diazepine; 4-methyl-1-(N-(2-methyl-4-(1-methyl-4,10-dihydropyrazolo[5,4-b][1,5]-benzodiazepin-5-ylcarbonyl)benzylcarbamoyl)-L-thioprolyl)perhydro-1,4-diazepine; 4,4-dimethyl-1-(N-(2-methyl-4-(1-methyl-4,10-dihydropyrazolo[5,4-b][1,5]-benzodiazepin-5-ylcarbonyl)benzylcarbamoyl)-L-thiprolyl)perhydro-1,4-diazepine; 4-methyl-1-(N-(2-methyl-4-(5,6,7,8-tetrahydrothieno[3,2-b]azepin-4-ylcarbonyl)-benzylcarbamoyl)-L-thioprolyl)perhydro-1,4-diazepine; 4-methyl-1-(N-(2-methyl-4-(5,6,7,8-tetrahydrothieno[3,2-b]azepin-4-ylcarbonyl)-benzyloxycarbonyl)-L-prolyl)perhydro-1,4-diazepine; (4R)-N^{α}-(2-chloro-4-(5,6,7,8-tetrahydrothieno[3,2-b]azepin-4-ylcarbonyl)benzyl-carbamoyl)-4-methoxy-L-proline-N-methyl-N-(2-picolyl)amide; or 1-((4R)-N^{α}-(2-chloro-4-(5,6,7,8-tetrahyd rothieno[3,2-b]azepin-4-ylcarbonyl)benzyl-carbamoyl)-4-methoxy-L-prolyl)-4-(1-pyrrolidinyl)piperidine; or a pharmaceutically acceptable salt thereof.

It is to be appreciated that structural embodiments described herein maybe combined together. Thus, for example, an embodiment described for formula 1, may also be applied in conjunction with any of the other possible combinable structural embodiments described for formula 1. Accordingly, this invention contemplates both individual embodiments as well as combinations of embodiments.

As used herein, the term “alkyl” is defined as lower alkyl radicals, having from 1 to 6 carbons. The alkyl radicals maybe straight chain, branched or C₃-C₆ cyclic. Some non-limiting examples of alkyl as defined herein include methyl, ethyl, propyl, iso-propyl, cyclopropyl, butyl, sec-butyl, pentyl, hexyl, cyclopentyl, and the like. The alkyl groups as defined herein may also be substituted with from 1-3 substituents selected from the group consisting of C₁₋₃ alkyl (unsubstituted), fluorine, chlorine, hydroxyl or phenyl.

The term acyl as defined herein refers to a (C═O)—R radical, where R is hydrogen, alkyl as defined previously, phenyl, naphthyl, pyridyl or thienyl, wherein said phenyl, naphthyl, pyridyl or thienyl are optionally substituted with from 1-3 groups selected from C₁₋₃ alkyl, halogen, O—C₁₋₃ alkyl, or OH. Some non-limiting examples of acyl are formyl, acetyl, benzoyl and the like.

The term “optionally substituted phenyl” as defined herein, refers to a phenyl radical wherein said phenyl radical can be substituted with from 1-3 substituents selected from the group consisting of C₁₋₃ alkyl, halogen, OH, and OC₁₋₃ alkyl.

This Invention relates to methods of treating mammals, preferably humans, for schizophrenia and schizophrenia-related disorders, which comprises the administration of a compound of formula 1 or 2. This invention also describes methods of treating mammals, preferably humans, for anxiety and anxiety-related disorders which methods include the administration of a compound of formula 1 or 2. This invention also describes methods of treating schizophrenia and schizophrenia-related disorders that comprise the administration of pharmaceutical compositions containing compounds of formula 1 or 2, wherein such compositions are administered to a mammal (preferably human). This invention also describes methods for treating mammals, preferably humans, for anxiety and anxiety-related disorders that comprise the administration of pharmaceutical compositions containing compounds of formula 1 or 2, or any of its structural embodiments described herein, or any of its structural embodiments described in any of the references that have been herein.

This invention also describes the use of a compound of formula 1 or 2 in the manufacture of medicaments for the treatment of schizophrenia or schizophrenia-related symptoms.

This invention also describes the use of a compound of formula 1 or 2 in the manufacture of a medicament for the treatment of anxiety and anxiety-related disorders.

Schizophrenia is typically diagnosed through the application of any of a number of commonly accepted criteria for the illness. Such definitions are provided by, for example, World Health Organization's International Statistical Classification of Diseases and Related Health Problems, or the American Psychiatric Association's Diagnostic and Statistical Manual of Mental Disorders (DSM), both of which are herein incorporated by reference in their entirety. In brief, schizophrenia is a disease which appears to have both environmental and genetic triggers, and which is typically defined by its manifest symptoms or behaviors including both positive (behaviors in addition to typical normal behaviors) and negative symptoms (behaviors retreating from normal behavior). Positive symptoms of schizophrenia include delusions, hallucinations, disorganized, excessive and often repetitive speech patterns, and disruptive or otherwise inappropriate conduct. Negative symptoms are usually typified by such behaviors as social withdrawal, lack of affect, tonal speech flatness, and reduced communicativeness. Besides symptoms associated with schizophrenia, people suffering from schizophrenia are often divided up into more general categories of behavior such as catatonic (immobile, non-responsive, rigid), disorganized schizophrenia (disorganized speech and behavior, and flat or inappropriate affect) or paranoid (suffering from delusions, often related to misperceived threats of persecution). For purposes of this invention, schizophrenia-related disorders refer to disorders wherein at least some of the symptoms of schizophrenia are present, although a classification of schizophrenia might not be appropriate. For example, brief psychotic disorder, schizophreniform disorder, schizoaffective disorder, and delusional disorder are all considered as schizophrenia-related disorders for purposes of this invention.

Anxiety can be generally described as a state of uneasiness or one of apprehension. Anxiety can demonstrate variations in cause, duration, etiology, appropriateness, etc, and it is generally accepted that probably all individuals suffer from anxiety at some time or another. Anxiety in its more serious forms can often paralyze an individual suffering from it, and acute or chronic, untreated anxiety can often lead to many severe physical and psychological disturbances. While anxiety maybe considered an appropriate response to dangerous or threatening situations, it also commonly occurs where the threat or perceived danger or threat is exaggerated or unfounded. Anxiety related disorders include panic disorder, agoraphobia, phobias (including social phobia), obsessive-compulsive disorder, acute stress disorder, post-traumatic stress disorder and generalized anxiety disorder.

As used herein, the term non-peptidergic means that the compounds so characterized do not contain two or more amino acids coupled together. Thus, for example, a non-peptidergic compound might contain one or more amino acid residues, but will not contain two amino acid residues coupled via an amide bond which links the C-terminus of one amino acid with the N-terminus of another amino acid. Amino acid as herein referred to refers to naturally occurring amino acids.

As used herein and in the appended claims, the singular forms “a,” “an,” and “the” include the plural reference unless the context clearly indicates otherwise. Thus, for example, a reference to “an oxytocin receptor agonist” includes a plurality of such oxytocin receptor agonists, and a reference to “a compound” is a reference to one or more compounds and equivalents thereof known to those skilled in the art, and so forth. Furthermore, an oxytocin agonist refers to a molecule as herein described and useful for the methods of this invention, wherein said molecule is capable of combining with, or otherwise modulating the oxytocin receptor and initiating an activity in a cell which is of the same qualitative type of activity as oxytocin itself would initiate, wherein said qualitative type of activity need be characterizable for only one or more measurable parameters. The type of response only need be qualitatively similar but does not have to meet a particular potency criteria. Thus, an agonist of this invention may behave like oxytocin on one or more parameters in one or more cells or tissues, but not necessarily for all parameters in all cells or tissues.

The abbreviations in the specification correspond to units of measure, techniques, properties, or compounds as follows: “min” means minutes, “h” means hour(s), “μL” means microliter(s), “mL” means milliliter(s), “mM” means millimolar, “M” means molar, “mmole” means millimole(s), “cm” means centimeters, “SEM” means standard error of the mean and “IU” means International Units.

In the context of this disclosure, a number of terms shall be utilized. The term “treatment” as used herein includes preventative (e.g., prophylactic), curative or palliative treatment and “treating” as used herein also includes preventative, curative and palliative treatment.

The term “effective amount,” as used herein, refers to an amount effective, at dosages, and for periods of time necessary, to achieve the desired result with respect to treatment of schizophrenia, schizophrenia-related disorders, anxiety, and anxiety-related disorders.

It will be appreciated that the effective amount of components of the present invention will vary from patient to patient not only with the particular compound, component or composition selected, the route of administration, and the ability of the components (alone or in combination with one or more combination drugs) to elicit a desired response in the individual, but also with factors such as the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage ultimately being at the discretion of the attendant physician. Dosage regimens may be adjusted to provide the improved therapeutic response. An effective amount is also one in which any toxic or detrimental effects of the components are outweighed by the therapeutically beneficial effects. Preferably, the compounds of the present invention are administered at a dosage and for a time such that the number and/or severity of the symptoms are decreased.

For example, for an afflicted patient, compounds of formula 1 or 2 may be administered, at a dosage of from about 0.1 mg/day to about 1000 mg/day, or about from 1 mg/day to about 500 mg/day or from about 10 mg/day to 500 mg/day for a time sufficient to reduce and/or substantially eliminate the number and/or severity of schizophrenic or anxiety related symptoms

The terms “component,” “composition of compounds,” “compound,” “drug,” or “pharmacologically active agent” or “active agent” or “medicament” are used interchangeably herein to refer to a compound or compounds or composition of matter which, when administered to a subject (human or animal) induces a desired pharmacological and/or physiologic effect by local and/or systemic action.

The term “modulation” refers to the capacity to either enhance or inhibit a functional property of a biological activity or process, for example, receptor binding or signaling activity. Such enhancement or inhibition may be contingent on the occurrence of a specific event, such as activation of a signal transduction pathway and/or may be manifest only in particular cell types.

“Administering,” as used herein, means either directly administering a compound or composition of the present invention, or administering a prodrug, derivative or analog which will form an equivalent amount of the active compound or substance within the body.

The term “subject” or “patient” refers to an animal including the human species that is treatable with the compositions, and/or methods of the present invention. The term “subject” or “subjects” is intended to refer to both the male and female gender unless one gender is specifically indicated. Accordingly, the term “patient” comprises any mammal which may benefit from treatment of schizophrenia, schizophrenia-related disorders, anxiety and anxiety-related disorders. Where the patient to be treated is a female of child-bearing years, it should be kept in mind that oxytocin receptor agonist activity is associated with labor induction in pregnant women and accordingly, this possible effect should be kept in mind when treating this population.

Some of the compounds of the present invention may contain chiral centers and such compounds may exist in the form of stereoisomers (i.e. enantiomers). The present invention includes all such stereoisomers and any mixtures thereof including racemic mixtures. Racemic mixtures of the stereoisomers as well as the substantially pure stereoisomers are within the scope of the invention. The term “substantially pure,” as used herein, refers to at least about 90 mole %, more preferably at least about 95 mole %, and most preferably at least about 98 mole % of the desired stereoisomer is present relative to other possible stereoisomers. Preferred enantiomers may be isolated from racemic mixtures by any method known to those skilled in the art, including high performance liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron, 33:2725 (1977); Eliel, E. L. Stereochemistry of Carbon Compounds, (McGraw-Hill, N.Y., 1962); Wilen, S. H. Tables of Resolving Agents and Optical Resolutions, p. 268 (E. L. Eliel, Ed., University of Notre Dame Press, Notre Dame, Ind. 1972).

The present invention includes prodrugs of the compounds of formula 1 or 2. “Prodrug,” as used herein, means a compound which is convertible in vivo by metabolic means (e.g. by hydrolysis) to a compound of formula 1 or 2. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs,” Textbook of Drug Design and Development, Chapter 5,113-191 (1991), Bundgaard, et al., Journal of Drug Deliver Reviews, 1992, 8:1-38, Bundgaard, J. of Pharmaceutical Sciences, 1988, 77:285 et seq.; and Higuchi and Stella (eds.) Prodrugs as Novel Drug Delivery Systems, American Chemical Society (1975).

Further, the compounds of formula 1 or 2 may exist in unsolvated as well as in solvated forms with pharmaceutically acceptable solvents such as water, ethanol, and the like. In general, the solvated forms are considered equivalent to the unsolvated forms for the purpose of the present invention.

The compounds of the present invention may be prepared in a number of ways well known to those skilled in the art. For example, the compounds of this invention maybe prepared by the methods disclosed in WO03/000692 and WO03/016316, both of which are herein incorporated by reference in their entirety.

In other embodiments, the invention is directed to pharmaceutical compositions, comprising:
a. at least compound of formula 1 or 2, or pharmaceutically acceptable salt thereof; and
b. at least one pharmaceutically acceptable carrier or excipient.

Generally, the compound of formula 1 or 2 or a pharmaceutically acceptable salt thereof will be present at a level of from about 0.1%, by weight, to about 90% by weight, based on the total weight of the pharmaceutical composition. In some embodiments, the compound of formula 1 or 2 or a pharmaceutically acceptable salt thereof will be present at a level of at least about 1%, by weight, based on the total weight of the pharmaceutical composition. In certain embodiments, the compound of formula 1 or 2 or a pharmaceutically acceptable salt thereof will be present at a level of at least about 5%, by weight, based on the total weight of the pharmaceutical composition. In still other embodiments, the compound of formula 1 or 2 or a pharmaceutically acceptable salt thereof will be present at a level of at least about 10%, by weight, based on the total weight of the pharmaceutical composition. In still yet other embodiments, the compound of formula 1 or 2 or a pharmaceutically acceptable salt thereof will be present at a level of at least about 25%, by weight, based on the total weight of the pharmaceutical composition.

Such compositions are prepared in accordance with acceptable pharmaceutical procedures, such as described in Remington's Pharmaceutical Sciences, 17th edition, ed. Alfonoso R. Gennaro, Mack Publishing Company, Easton, Pa. (1985). Pharmaceutically acceptable carriers are those that are compatible with the other ingredients in the formulation and biologically acceptable.

The compounds of this invention may be administered orally or parenterally, neat or in combination with conventional pharmaceutical carriers. Applicable solid carriers can include one or more substances that may also act as flavoring agents, lubricants, solubilizers, suspending agents, fillers, glidants, compression aids, binders or tablet-disintegrating agents or an encapsulating material. In powders, the carrier is a finely divided solid that is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers may be used in preparing solutions, suspensions, emulsions, syrups, and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols e.g. glycols) and their derivatives, and oils (e.g. fractionated coconut oil and arachis oil). For parenteral administration the carrier can also be an oily ester such as ethyl oleate and isopropyl myristate. Sterile liquid carriers are used in sterile liquid form compositions for parenteral administration.

Liquid pharmaceutical compositions, which are sterile solutions or suspensions, can be administered by, for example, intramuscular, intraperitoneal or subcutaneous injection. Sterile solutions can also be administered intravenously. Oral administration may be either liquid or solid composition form.

In some embodiments, the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example packeted powders, vials, ampoules, prefilled syringes or sachets containing liquids. The unit dosage form can be, for example, a capsule or tablet itself, or it can be the appropriate number of any such compositions in package form.

In another embodiment of the present invention, the compounds useful in the present invention may be administered to a mammal with one or more other pharmaceutical active agents such as those agents being used to treat any other medical condition present in the mammal. Examples of such pharmaceutical active agents include tranquilizers, anti-psychotics, anti-depressants, and the like.

The one or more other pharmaceutical active agents may be administered in a therapeutically effective amount simultaneously (such as individually at the same time, or together in a pharmaceutical composition), and/or successively with one or more compounds of the present invention.

The route of administration may be any route, which effectively transports the active compound of formula 1 or 2 to the appropriate or desired site of action, such as oral, nasal, pulmonary, transdermal, such as passive or iontophoretic delivery, or parenteral, e.g. rectal, depot, subcutaneous, intravenous, intraurethral, intramuscular, intranasal, ophthalmic solution or an ointment. Furthermore, the administration of compound of formula 1 with other active ingredients may be concurrent or simultaneous.

### EXAMPLES

Oxytocin Receptor Agonists As Anxiolytic-like Agents

### METHODS AND MATERIALS

Animals: Male Swiss-Webster mice weighing 18-24 g were housed in groups of 15 in hanging wire cages, allowed access to food and water ad libitum, and maintained on a 12-hour light dark cycle. All behavioral testing was performed during the light cycle. All studies were previously approved by the Institutional Animal Care and Use Committee, and performed in accordance to the Guide for the Care and Use of Laboratory Animals as adopted and promulgated by the National Institutes of Health.

Test Compounds: Oxytocin (American Peptide Company, Sunnyvale, Calif.) was dissolved in a saline vehicle. The oxytocin agonist 4,-(3,5-Dihydroxy-benzyl)-piperazine-1-carboxylic acid 2-methyl-4-(3-methyl-4,10-dihydro-3H-2,3,4,9-tetra-aza-benzo[f]azulene-9-carbonyl)-benzylamide (hereinafter “cpd A”) and oxytocin antagonist 10-[(2-Methyl-2′-trifluoromethyl-[1,1′-biphenyl]-4-yl)carbonyl]-10,11-dihydro-5H-pyrrolo[2,1-c][1,4]benzodiazepine-3-carboxylic acid-bis-(2-hydroxy-ethyl)-amide (hereinafter “cpd B”) (Patent W/O 02/083680) were prepared and dissolved in a 1% Tween-80/1% DMSO/saline vehicle.

4,-(3,5-Dihydroxy-benzyl)-piperazine-1-carboxylic acid 2-methyl-4-(3-methyl-4,10-dihydro-3H -2,3,4,9-tetra-aza-benzo[f]azulene-9-carbonyl)-benzylamide hydrochloride was prepared by dissolution of 4,-(3,5-Dihydroxy-benzyl)-piperazine-1-carboxylic acid 2-methyl-4-(3-methyl-4,10-dihydro-3H-2,3,4,9-tetra-aza-benzo[f]azulene-9-carbonyl)-benzylamide (4.2 g) in EtOH (100 mL) and the solution was cooled with an ice bath. Hydrochloric acid was bubbled into the solution for 10 min. Ether was added and the resulting white precipitate was collected by filtration to give 2.4 g of the title compound. MS (ES) m/z [M-H] 580.3

ICV injections: Mice were lightly anesthetized with halothane. Oxytocin was administered into either the left or right ventricle by visual location. A 26 gauge Hamilton syringe with 3 mm needle was used for injections and the injection site was visualized by locating the middle of the invisible line that runs diagonally from the left eye to the right ear. Test compounds were injected in a 2 μl total volume.

Four-Plate Test (FPT:) The four-plate apparatus consists of a Plexiglas chamber (18×25×16 cm) floored with four identical rectangular metal plates (8×11 cm), which are separated from one another by a gap of 4 mm and connected to a computerized device that can deliver electric shocks (0.8 mA, 0.5 sec) (Aron et al. Evaluation of a rapid technique for detecting minor tranquilizers, Neuropharmacology 10: 459-69 (1971)). In this test, mice are placed into the chamber and following a brief (18 seconds) habituation period, the animal's innate motivation to explore the novel environment is suppressed by the delivery of a mild foot shock every time the animal crosses any of the boundaries (gaps) while moving from one plate to another (referred to as a ‘punished crossing’). Following any punished crossing, there is a 3-second time out where the mouse may cross the electric plates without receiving another shock. An experimenter blind to the dosing conditions administers shocks, and a computer records the total number of punished crossings an animal makes during a 1-minute testing period. Clinically effective classes of anxiolytic compounds such as benzodiazepines, selective serotonin reuptake inhibitors (SSRIs), or 5-HT_{1A} antagonists produce increases in punished crossings in this paradigm, which is indicative of anxiolytic-like activity (Aron et al. Evaluation of a rapid technique for detecting minor tranquilizers. Neuropharmacology 10: 459-69 (1971); Bourin et al. Comparison of behavioral effects after single and repeated administrations of four benzodiazepines in three mice behavioral models. J Psychiatry Neurosci 17: 72-7 (1992); Hascoet et al. Anxiolytic-like effects of antidepressants after acute administration in a four-plate test in mice. Pharmacol Biochem Behav 65: 339-44 (2000)). In all experiments, the testing procedure consisted of either a single injection or two injections followed by a test session 30 minutes later.

Statistical Analysis: One-way analysis of variance (ANOVA) was performed to determine effects of test compound treatments, followed by Least Significant Difference Tests for a post hoc analysis. All figures are shown with average ±SEM.

RESULTS

Oxytocin Produces Anxiolytic-like Effects in the Mouse FPT

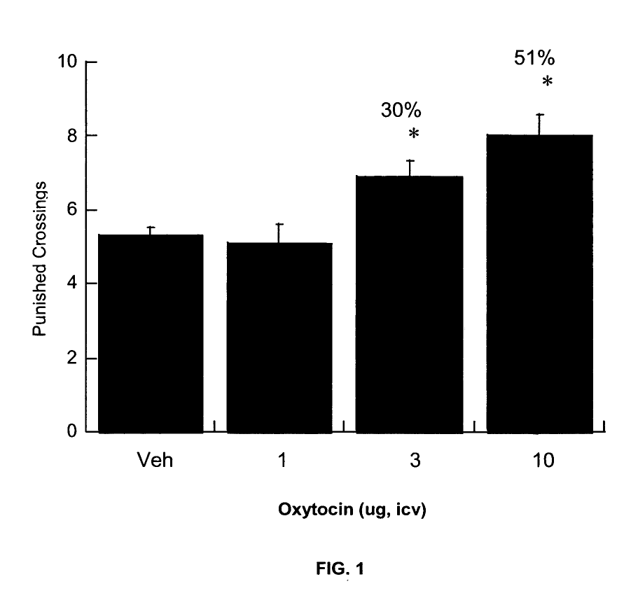

The mouse FPT is a frequently used preclinical model for detecting anxiolytic activity of test compounds. Central administration of oxytocin (1-10 mg, icv) produced a dose-dependent increase in punished crossings (F_{(3.36)}=8.99, p<0.0001; FIG. 1). Post hoc analysis revealed significant increases in punished crossings at the two highest doses (30% and 51% increase from vehicle for 3 and 10 μg respectively; p<0.05). This data suggests an anxiolytic-like effect of centrally administered oxytocin.

The Oxytocin Agonist Produces Anxiolytic-like Effects in the Mouse FPT

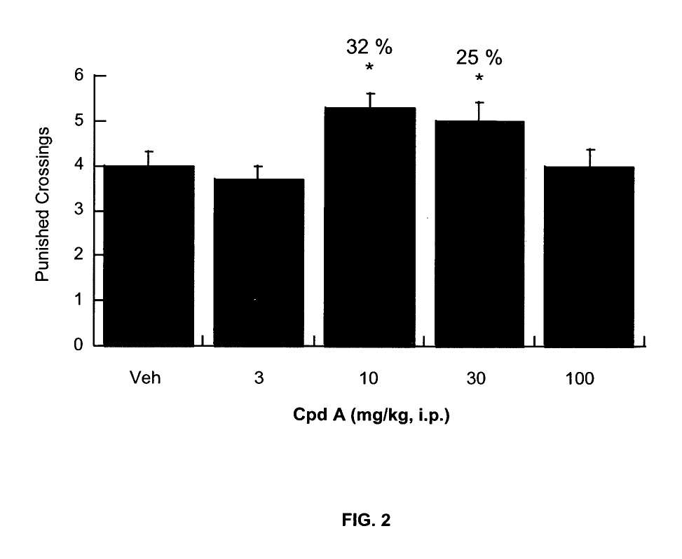

Peripheral administration of cpd A (3-100 mg/kg, ip), produced a significant overall effect on punished crossings (F(4,45)=4.11, p<0.01; FIG. 2). Post hoc analysis revealed significant increases in punished crossings in the 10 and 30 mg/kg group (32% and 25% increase from vehicle for 10 and 30 mg/kg respectively; p<0.05).). This data suggests an anxiolytic-like effect of peripherally administered cpd A.

Blockade of the anxiolytic-like effects of cpd A by a brain-penetrant oxytocin receptor antagonist

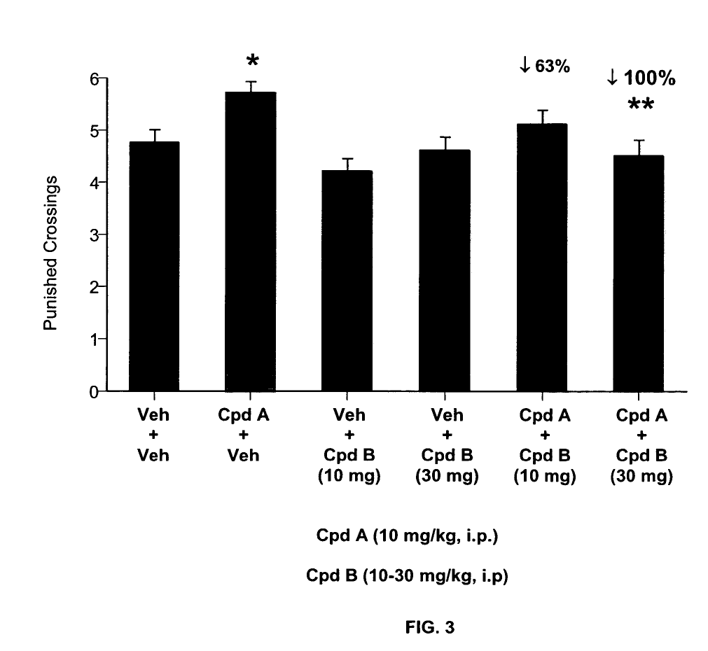

To determine if the anxiolytic-like effects of cpd A were mediated by the oxytocin receptor (OTR), cpd B, a brain-penetrant OTR antagonist, was administered in combination with cpd A. Cpd A (10 mg/kg, ip) increased punished crossings compared to vehicle (p<0.05, FIG. 3). Co-administration of cpd B (10-30 mg/kg, ip) blocked the anxiolytic-like effect of cpd A in a dose-dependent manner (63% and 100% reversal for 10 and 30 mg/kg respectively), which reached significance at the 30 mg/kg dose p<0.05). Cpd B had no effect on punished crossings when administered alone. This data indicates that the OTR antagonist cpd B blocks the anxiolytic-like effect of cpd A in the four-plate model.

Oxytocin Receptor Agonists As Antipsychotics

### METHODS AND MATERIALS

Prepulse inhibition of the acoustic startle reflex (PPI) is an operational measure of sensorimotor gating that can be measured across many species. Deficits in PPI have been reported in patients with schizophrenia, leading to its use as a preclinical model of the disease. In rats, PPI is decreased in a manner homologous to that seen in schizophrenia following administration of certain psychotomimetic drugs (e.g. MK801; amphetamine). In our study we utilized MK801, a non-competitive NMDA antagonist and d-Amphetamine, a non-selective dopamine agonist. MK801 (0.1 mg/kg sc, 10 min prior to test) and d-Amphetamine (4 mg.kg sc, 10 min prior to test) produced significant disruption across three prepulse intensities (5 dB, 10 dB & 15 dB).

Animals: Male Sprague-Dawley derived Rats (SD) weighing 200-250 g were group housed in standard bedding cages, allowed access to food and water ad libitum, and maintained on a 12-hour light dark cycle. All behavioral testing was performed during the light cycle. All studies were previously approved by the Institutional Animal Care and Use Committee, and performed in accordance to the Guide for the Care and Use of Laboratory Animals as adopted and promulgated by the National Institutes of Health.

Test Compounds: The oxytocin agonist cpd A was dissolved in a 1% Tween-80/1% DMSO/saline vehicle. MK801 (Sigma, St. Louis, Mo.) was dissolved in 2% Tween-80/saline. d-Amphetamine (Sigma, St. Louis, Mo.) was dissolved in saline.

Test Equipment

Each testing chamber (SR-LAB system, San Diego Instruments) consisted of a Plexiglas cylinder (8.8 cm in diameter) mounted on a frame and held in position by four metal pins to a base unit. Movement of the rat within the cylinder was detected by a piezoelectric accelerometer attached below the frame. A loudspeaker mounted 24 cm above the cylinder provided background white noise, acoustic noise bursts and acoustic prepulses. The entire apparatus was housed in a ventilated enclosure (39×38×56 cm). Presentation of acoustic pulse and prepulse stimuli were controlled by the SR-LAB software and interface system, which also digitized, rectified and recorded the responses from the accelerometer. Mean startle amplitude was determined by averaging 100, 1 ms readings taken from the beginning of the pulse stimulus onset. For calibration purposes, sound levels were measured with a Quest sound level meter, scale “A”, with the microphone placed inside the Plexiglas cylinder.

Test Sessions

Test sessions began when the rats were placed in the startle chambers for a 5-min acclimation period with a 64 dB (A) background of white noise. After the acclimation period, rats were exposed to four types of stimuli. The startle-eliciting stimulus was a 20-ms broad band burst at a sound pressure level of 120 dB (A). Three different intensities of auditory prepulse stimuli were utilized. These consisted of a 69, 74 or 79 dB (A), 20-ms broad band burst which was presented 100-ms (onset to onset), prior to the startle pulse. These four trial types were presented against a constant 64 dB (A) background of white noise. A test session consisted of an initial pulse stimulus, followed by 15 sequences of the four stimulus types, presented in pseudorandom order, for a total of 61 trials. Inter-trial intervals averaged 15 s.

Evaluation of Results

Startle amplitude was defined as the mean value of pulse alone trials. To evaluate the effect of drug treatment on startle response, data from the pulse alone trials was analyzed using one-factor ANOVA with repeated measures (one-way randomized block design), followed by a least significant difference (LSD) post-hoc test (comparison was made to vehicle/disrupting agent control). Prepulse inhibition was defined as 100-[(startle amplitude on prepulse trials/startle amplitude on pulse alone trials)×100]. Although data for gating at three different prepulse intensities was generated, an averaged gating score across all prepulse intensities was calculated and this was analyzed by one factor ANOVA with repeated measures (one-way randomized block design). This was followed by a LSD post-hoc. The criterion for significance for alterations in both startle amplitude and PPI was set at P<0.05.

Results

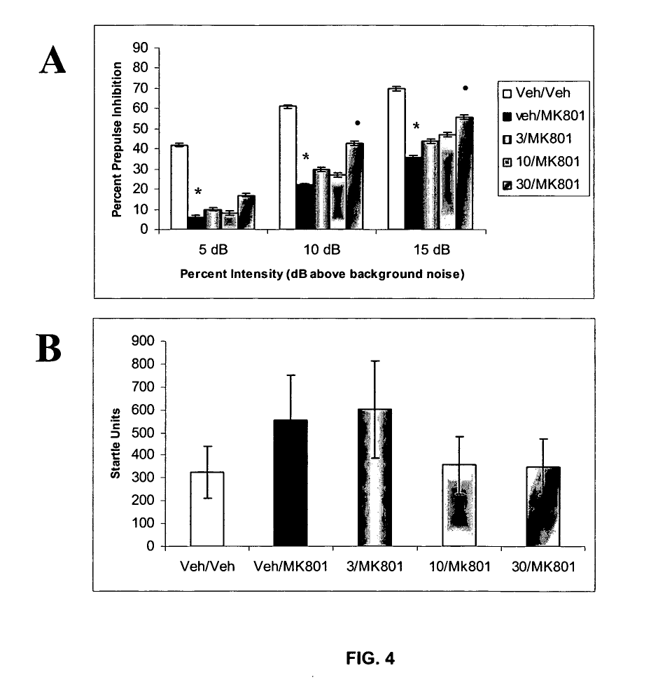

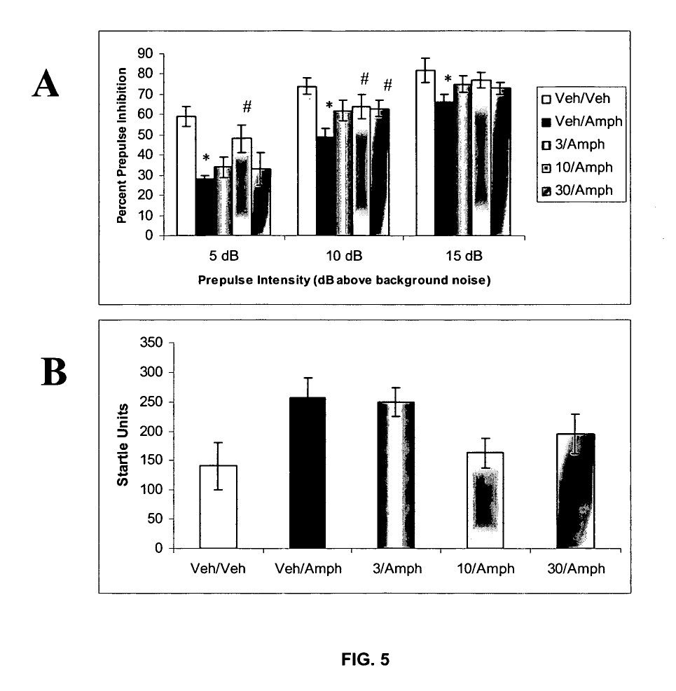

We observed that oxytocin (0.04-1 mg/kg s.c.) reversed the MK801 induced deficits in PPI in rats in a dose dependent manner (data not shown). This observation is in agreement with published observations (Feifel & Reza, Oxytocin modulates psychotomimetic-induced deficits in sensomotor gating, Psychopharmacology (Berl) 141(1):93-8 (1999) ). Oxytocin has been implicated to play an important role in modulation of dopaminergic and glutamergic regulation of PPI and thus oxytocin may act as a novel endogenous antipsychotic agent. We observed that MK801, a non-competitive NMDA antagonist (0.1 mg/kg s.c., 10 min prior to test) produced significant disruption in PPI across three prepulse intensities (TreatmentXPPI interaction, p <0.05, FIG 4B) with no effect on startle alone (p >0.05, FIG. 4B). Cpd A (3-30 mg/kg, i.p.), a non-peptide agonist of the OTR reversed MK801 induced deficits in PPI at 10 dB & 15 dB levels at the highest dose tested (30 mg/kg) (FIG. 4A). d-Amphetamine, a non-selective dopamine agonist, (4 mg/kg s.c., 10 min prior to test) produced significant disruption across all three prepulse intensities (TreatmentXPPI interaction, p <0.05, FIG. 5B). Cpd A (HCl salt) (10 mg/kg, i.p.), a non-peptide agonist of the OTR reversed d-amphetamine induced disruption at 5 dB and 10 dB; Cpd A (HCl salt) at 30 mg/kg, i.p. reversed the d-amphetamine induced disruption at 10 dB (FIG. 5A). Collectively, this evidence suggests the clinical utility of OTR agonists as antipsychotics.

When ranges are used herein for physical properties, such as molecular weight, or chemical properties, such as chemical formulae, all combinations and subcombinations of ranges specific embodiments therein are intended to be included.

The disclosures of each patent, patent application and publication cited or described in this document are hereby incorporated herein by reference, in its entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A method of treating schizophrenia or a schizophrenia-related disorder, anxiety or an anxiety-related disorder comprising the administration to a mammal a compound of formula 1, or a pharmaceutically acceptable salt thereof: 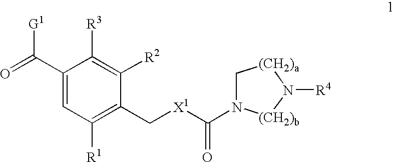
wherein:
G¹ is 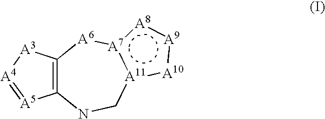
wherein A₃ is S; NH; N—C₁₋₃alkyl; —CH═CH— or CH═N; A₄ is CH; A₅ is CH; A₆ is NH; A₇ is C; A₈ is N—(CH₂)_{d}-R₇; A₉ is N; A₁₀ is CH and A₁₁ is C; wherein d is 1, 2 or 3; and R₇ is selected from hydrogen; C₁₋₃ alkyl; optionally substituted phenyl; OH; O-alkyl; O-acyl; S-alkyl; NH₂; NH-alkyl; N(alkyl)₂; NH-acyl; N(alkyl)-acyl; CO₂H; CO₂-alkyl; CONH₂; CONH-alkyl; CON(alkyl)₂; CN; and CF₃;
R¹, R² and R³ are each independently selected from hydrogen; alkyl; Fl or Cl;
a is 1 or 2;
b is 1, 2 or3;
X¹ is O or NH; and
R⁴ is selected from

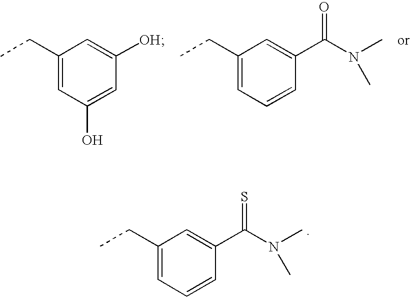

2. The method of claim 1, wherein G¹ is

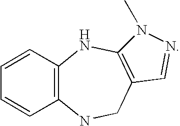

3. The method of claim 1, wherein R² is methyl.

4. The method of claim 1, wherein R⁴ is

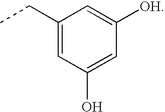

5. The method of claim 1, wherein X¹ is NH.

6. The method of claim 1, wherein the compound of formula 1 is:
a) 4,-(3,5-Dihydroxy-benzyl)-piperazine-1-carboxylic acid 2-methyl-4-(3-methyl-4,10-dihydro-3H-2,3,4,9-tetra-aza-benzo[f]azulene-9-carbonyl)-benzylamide;
b) 4,-(3, 5-Dihydroxy-benzyl)-piperazine-1-carboxylic acid 2,6-dimethyl -4-(3-methyl-4,10-dihydro-3H-2,3,4,9-tetra-aza-benzo[f]azulene-9-carbonyl)-benzylamide;
c) 4,-(3,5-Dihydroxy-benzyl)-piperazine-1-carboxylic acid 3-chloro-4-(3-methyl -4,10-dihydro-3H-2,3,4,9-tetra-aza-benzo[f]azulene-9-carbonyl)-benzylamide;
d) 4,-(3,5-Dihydroxy-benzyl)-piperazine-1-carboxylic acid 2-fluoro-4-(3-methyl -4,10-dihydro-3H-2,3,4,9-tetra-aza-benzo[f]azulene-9-carbonyl)-benzylamide;
e) 4,-(3-Dimethylcarbamoyl-benzyl)-piperazine-1-carboxylic acid 2-methyl -4-(3-methyl-4,10-dihydro-3H-2,3,4,9-tetra-aza-benzo[f]azulene-9-carbonyl)-benzylamide; and
f) 4,-(3-Dimethylthiocarbamoyl-benzyl)-piperazine-1-carboxylic acid 2-methyl-4-(3-methyl-4,10-dihydro-3H-2,3,4,9-tetra-aza-benzo[f]azulene-9-carbonyl)-benzylamide; or a pharmaceutically acceptable salt thereof.

7. The method of claim 1, wherein the compound of formula 1 is administered with at least one pharmaceutically acceptable excipient.

8. The method of claim 1, wherein the mammal is a human.

9. A method of treating schizophrenia or a schizophrenia-related disorder, anxiety, or an anxiety related disorder, comprising the administration to a mammal a compound of formula 2, or a pharmaceutically acceptable salt thereof: 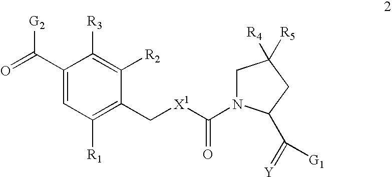
wherein:
G₂ is (II) 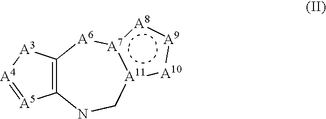
wherein A₃ is S; NH; N—C₁₋₃alkyl; —CH═CH— or CH═N; A₄ is CH; A₅ is CH; A₆ is NH; A₇ is C; A₈ is N—(CH₂)_{d}-R₇; A₉ is N; A₁₀ is CH and A₁₁ is C; wherein d is 1, 2 or 3; and R₇ is selected from hydrogen; C₁₋₃ alkyl; optionally substituted phenyl; OH; O-alkyl; O-acyl; S-alkyl; NH₂; NH-alkyl; N(alkyl)₂; NH-acyl; N(alkyl)-acyl; CO₂H; CO₂-alkyl; CONH₂; CONH-alkyl; CON(alkyl)₂; CN; and CF₃;
R₁, R₂, and R₃ are each independently selected from the group consisting of hydrogen; alkyl; O-alkyl; Fl; Cl; or Br;
X₁ is NH or O;
R₄ and R₅ are each independently selected from the group consisting of hydrogen; O-alkyl; O-benzyl; and F; or R₄ and R₅ together are =O; —O(CH₂)ₐO—; or
—S(CH₂)ₐS—;
a is 2 or 3;
Y is O or S; and
G₁ is 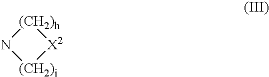
wherein h is 1 or 2; I is 1, 2 or 3; and X₂ is N-alkyl.

10. The method according to claim 9, wherein G₂ is:

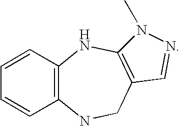

11. The method according to claim 9, wherein two of R¹, R² and R³ are hydrogen and the other is not hydrogen.

12. The method according to claim 9, wherein X₁ is NH.

13. The method according to claim 9, wherein R₄ and R₅ are each independently selected from hydrogen and O-alkyl.

14. The method according to claim 9, wherein G₁ is 1-Methyl-[1,4]diazepane.

15. The method according to claim 8, wherein the compound of formula 2 is:
a) 4-methyl-1-(N-(2-methyl-4-(2,3,4,5-tetrahydro-1,5-benzodiazepin-4-on-1-yl-carbonyl)benzylcarbamoyl)-L-thioprolyl)perhydro-1,4-diazepine;
b) 4-methyl-1-(N-(2-methyl-4-(1-methyl-4,10-dihydropyrazolo[5,4-b][1,5]-benzodiazepin-5-ylcarbonyl)benzylcarbamoyl)-L-thioprolyl)perhydro-1,4-diazepine;
c) 4,4-dimethyl-1-(N-(2-methyl-4-(1-methyl-4,10-dihydropyrazolo[5,4-b][1,5]-benzodiazepin-5-ylcarbonyl)benzylcarbamoyl)-L-thiprolyl)perhydro-1,4-diazepine;
d) 4-methyl-1-(N-(2-methyl-4-(5,6,7,8-tetrahydrothieno[3,2-b]azepin-4-ylcarbonyl) -benzylcarbamoyl)-L-thioprolyl)perhydro-1,4-diazepine;
e) 4-methyl-1-(N-(2-methyl-4-(5,6,7,8-tetrahydrothieno[3,2-b]azepin-4-ylcarbonyl) -benzyloxycarbonyl)-L-prolyl)perhydro-1,4-diazepine;
f) (4R)-N^{α}-(2-chloro-4-(5,6,7,8-tetrahyd rothieno[3,2-b]azepin-4-ylcarbonyl)benzyl-carbamoyl) -4-methoxy-L-proline-N-methyl-N-(2-picolyl)amide; or
g) 1-((4R)-N^{α}-(2-chloro-4-(5,6,7,8-tetrahydrothieno[3,2-b]azepin-4-ylcarbonyl) benzyl-carbamoyl)-4-methoxy-L-prolyl)-4-(1-pyrrolidinyl)piperidine;
or a pharmaceutically acceptable salt thereof.

16. The method of claim 9, wherein the compound of formula 2 is administered with at least one pharmaceutically acceptable excipient.

17. The method of claim 9, wherein the mammal is a human.
